(19) United States
(12) Patent Application Publication
(10) Pub. No.: US 2025/0090820 A1
(43) Pub. Date: Mar. 20, 2025

(54) LOCALIZED DRUG DELIVERY TO PREVENT VASCULAR RESTENOSIS

(71) Applicant: Ohio State Innovation Foundation, Columbus, OH (US)

(72) Inventors: John LANNUTTI; Marco COUTINHO DA SILVA, Worthington, OH (US)

(21) Appl. No.: 18/727,272

(22) PCT Filed: Jan. 9, 2023
(86) PCT No.: PCT/US2023/060325

§ 371 Date: Jul. 8, 2024

Related U.S. Application Data
(60) Provisional application No. 63/297,428, filed on Jan. 7, 2022.

(51) Int. Cl. A61M 31/00; A61L 31/04; A61L 31/14; A61L 31/16

(52) U.S. Cl. CPC A61M 31/002; A61L 31/041; A61L 31/146; A61L 31/148; A61L 31/16; A61L 2300/416; A61M 2207/00; A61M 2210/12

(57) ABSTRACT

Described are drug delivery devices, methods of making and using the devices. The drug delivery device can include a cuff having a hollow shape with two closed ends. The cuff can include an inner wall, an outer wall, a lumen extending therethrough, and a composition including an active agent encapsulated within a carrier including a hydrophobic polymer, hydrophilic polymer, amphiphilic polymer, copolymer, or blends thereof present within the lumen.

### CROSS-REFERENCE TO RELATED APPLICATIONS

The application claims the benefit of U.S. Provisional Application No. 63/297,428, filed Jan. 7, 2022, which is hereby incorporated herein by reference in its entirety.

### STATEMENT REGARDING FEDERALLY SPONSORED RESEARCH OR DEVELOPMENT

This invention was made with government support under L15AC00146 awarded by the Bureau of Land Management. The government has certain rights in the invention.

### BACKGROUND

Vascular restenosis is a billion-dollar medical problem in the United States. Coronary artery bypass grafting is a widely practiced intervention in the treatment of coronary artery disease. Stents, tissue engineered grafts and healthy blood vessels transplanted from the chest or legs are used to bypass clogged or partially clogged arteries around the heart. Restenosis—or clogging of these arteries—over time can result in a loss of patency even in transplanted arteries. Drug-loaded polymer formulations are a rational technique to deliver compounds locally for a prolonged period of time to the vessel wall to inhibit intimal hyperplasia. A substantial disadvantage of these methods is that they are water-based, which restricts the half-life of the delivery system. A cuff made of a polymer suitable for eluting anti-restenotic compounds would be an important step towards a useful preventive measure. Poly(-caprolactone) (PCL) is a biocompatible and biodegradable polymer and PCL formulations have already been investigated as stent eluting coatings for paclitaxel in a rabbit model of restenosis and in the Boston Scientific DES program (TAXUS™). Polymeric formulations consisting of PCL blended with poly(ethylene glycol) (PEG) have been developed in the past for local delivery of anti-oncogenic drugs. The relatively hydrophilic PEG dissolves into the aqueous medium and open channels within the PCL matrix through which water can penetrate and drugs can be sustainably diffused out. Unfortunately, paclitaxel and rapamycin-eluting PCL cuffs only provide sustained drug release for approximately 3 weeks. In contrast, issues with neointimal hyperplasia of these grafts can occur for years following implantation. Sustained release for such periods of months or years could result in substantially reduced neointima formation for any given anti-restenotic agent ideally without systemic adverse effects or effect on cuffed arteries. What is envisioned is a drug delivery technique involved shaped, hollow polymeric cylinders or cuffs that surround the transplant (graft, stent or transplanted artery) to slowly deliver anti-restenosis drugs—such as rapamycin and paclitaxel—for extended periods of time (months to years). Such extended delivery times are enabled by the use of: (1) hydrophobic or semi-hydrophobic carriers that preserve the bioactivity of the drugs being delivered; (2) engineered porosity that can slowly meter out the compounds of interest and (3) a relatively high drug volume that can sustain drug concentrations high enough to create continuous delivery processes over long time periods.

The compositions and methods disclosed herein address these and other needs.

### SUMMARY

Described herein are drug delivery devices including a cuff having a hollow shape with two closed ends. The cuff can include an inner wall, an outer wall, a lumen extending therethrough, and a composition including an active agent encapsulated within a carrier including a hydrophobic polymer, hydrophilic polymer, amphiphilic polymer, copolymer, or blends thereof present within the lumen. The inner wall and the outer wall can include an inner surface and an outer surface. The inner surface of the inner and outer walls can surround the lumen. The outer surface of the inner wall can surround a hollow center. The inner and outer walls can include a biodegradable polymer. In some embodiments, the biodegradable polymer can include polycaprolactone (PCL).

In some embodiments, the inner, outer, or inner and outer wall can further include a non-biodegradable polymer. In some embodiments, the non-biodegradable polymer can include polyethylene terephthalate (PET).

In some embodiments, the cuff can have a pore size of from 100 nm to 5 μm. In some embodiments, the active agent can be in the form of a solid. In some embodiments, the cuff can have a length of from 0.1 cm to 20 cm. In some embodiments, the cuff can have an inner wall diameter of from 100 μm to 3000 μm. In some embodiments, the cuff can have an outer wall diameter from 50 μm to 300 μm greater than the inner wall diameter. In some embodiments, the cuff can surround a transplant graft, stent, or transplanted artery.

In some embodiments, the active agent can include an anti-restenosis drug. In some embodiments, the active agent can be present in the composition in an amount of from 1 μg/ml to 100,000 μg/ml. In some embodiments, the cuff can release the active agent over a period of at least 30 days.

Described are also methods for preparing a drug delivery device described herein. The method can include forming the inner wall of the cuff on a first rod; forming the outer wall of the cuff on a second rod, wherein the diameter of the second rod is from 50 μm to 300 μm greater than the diameter of the first rod, wherein forming the inner wall and outer wall can include electrospinning using a solution of a biodegradable polymer, a porogen, and optionally a non-biodegradable polymer and a voltage difference of 10 kV to 30 kV; removing the porogen from the inner wall, outer wall, or any combination thereof; sintering the inner wall and outer wall; injecting a composition including the active agent encapsulated within a carrier into the lumen of the cuff; and closing the inner wall and the outer wall together to form the drug delivery device.

In some embodiments, sintering can include heating at a temperature of from 50° C. to 150° C. In some embodiments, sintering can include heating for a period of from 1 minute to 6 hours.

Described herein are also methods of treating restenosis in a subject including administering to the subject in need thereof an effective amount of an active agent using a drug delivery device described herein.

Described herein are also methods of inhibiting intimal hyperplasia in a subject including administering to the subject in need thereof an effective amount of an active agent using a drug delivery device described herein.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIGS. 1A-1D show the initial structure post-electrospinning.

FIGS. 2A-2C shows a capsule formation at scale (2A) goal structure, (2B) as spun, (2C) post-sintered.

FIG. 3 shows model compounds (Rhodamine B (RhB), Rose Bengal (RB), and BSA-FITC) used in the Examples.

FIG. 4 shows results for RhB release from as-spun fiber capsules.

FIGS. 5A-5H show images of microstructural transformations at different temperatures (5A and 5B) at 56° C., (5C and 5D) at 57° C., (5E and 5F) at 58° C., (5G and 5H) at 59° C.

FIGS. 6A-6B show progressive elimination of porosity as temperature increases. (6A) shrinkage as temperature increases. (6B) average pore area as temperature increases.

FIG. 7 shows a graph of percent RhB release of 200 μg versus time.

FIG. 8 shows a graph of percent RB release of 200 μg versus time.

FIG. 9 shows a graph of percent BSA-FITC release of 200 μg versus time.

FIG. 10 shows the effects of thickness on percent release of RhB.

FIGS. 11A-11B show images comparing porosity.

FIGS. 12A-12D show images of HEPES salt and the effects of controlled salt confinement for controlled porosity using different HEPES salt concentration: (12A) 5% HEPES, (12B) 10% HEPES, (12C) 20% HEPES, and (12D) 20% HEPES prior to leaching.

FIGS. 13A-13F show images of initial fibers and controlled salt confinement for controlled porosity following sintering when HEPES salt is present (13A) 80:10:10 as spun, (13B) 80:10:10 water treated fibers, (13C) 80:10:10 sintered, (13D) 80:10:10 sintered and water treated, (13E) water-treated 88:10:2, and (13F) water-treated 70; 10:20.

FIG. 14 shows structures of sample oil carriers to that could used to prevent or minimize hydrolytic degradation of the drug of interest.

FIG. 15 shows release rate (into PBS) of Rose Bengal using different carriers.

FIG. 16 shows release rate (into PBS) of Rose Bengal using DBE-224 as the carrier capsules with different ratios of PCL:PET:HEPES making up the wall of said capsules.

FIG. 17 shows water infusion versus time for different capsules using different oil carriers. Note that hydrophobic silicone oil prevents any water pickup over the 4 month period even when porosity is present in the wall.

FIG. 18 shows a conceptual view of release via saltatory diffusion between the embedded pores and the surrounding polymer.

FIG. 19 shows a graph of the weight gain/loss(%) versus time for PLGA (85 G/15 L).

FIG. 20 shows images in vitro and in vivo studies at 37° C. and PBS for 85:15 PLGA release and PCL:PET 75:25 implantation in the horse.

FIG. 21 shows a drug delivery device 100 including a cuff 101 having a hollow cylindrical shape with two closed ends (102a and 102b). The cuff including an inner wall 103, an outer wall 104, a lumen 105 extending therethrough, and a composition described herein within the lumen 105.

FIG. 22 shows a top-down view of a drug delivery device 100 including a cuff 101 having a hollow cylindrical shape with two closed ends (102a and 102b). The cuff including an inner wall 103, an outer wall 104, a lumen 105 extending therethrough, and a composition described herein within the lumen 105. The inner wall 103 can include an inner surface 103a and an outer surface 103b and the outer wall 104 can include an inner surface 104a and an outer surface 104b. The inner surface (103a and 104a) of the inner and outer walls (103 and 104) surrounds the lumen 105. The outer surface 103b of the inner wall 103 surrounds a hollow center 106.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

The term “comprising” and variations thereof as used herein is used synonymously with the term “including” and variations thereof and are open, non-limiting terms and do not exclude additional elements or steps. Although the terms “comprising” and “including” have been used herein to describe various embodiments, the terms “consisting essentially of” and “consisting of” can be used in place of “comprising” and “including” to provide for more specific embodiments of the invention and are also disclosed. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of“one or more,” “at least one,” and “one or more than one.” Therefore, an element preceded by “a” or “an” does not, without more constraints, preclude the existence of additional identical elements. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraocular, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a natural or synthetically derived chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, a symptom of a disease or disorder, or preventing or altering a physiological process. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial agent or agents can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced or increased as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like, and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include companion or domesticated animals (e.g., cats, dogs, horses etc.), livestock (e.g., cattle, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

Reference will now be made in detail to specific aspects of the disclosed materials, compounds, compositions, articles, and methods, examples of which are illustrated in the accompanying Examples and Figures.

### Drug Delivery Device

Disclosed are drug delivery devices including a cuff having a hollow shape with two closed ends; the cuff including an inner wall, an outer wall, a lumen extending therethrough and a composition including an active agent encapsulated within a carrier within the lumen. In some embodiments, the inner wall and the outer wall can include an inner surface and an outer surface. In some embodiments, the inner surface of the inner and outer walls can surround the lumen. In some embodiments, the outer surface of the inner wall surrounds a hollow center. In some embodiments, the inner and outer walls can include a biodegradable polymers. In some embodiments, the matrix can have any suitable geometry including, but not limited to, tubular, cylindrical, hexagonal, square, square tubular, hexagonal tubular, cylindrical tubular. In some embodiments, the matrix can be cylindrical.

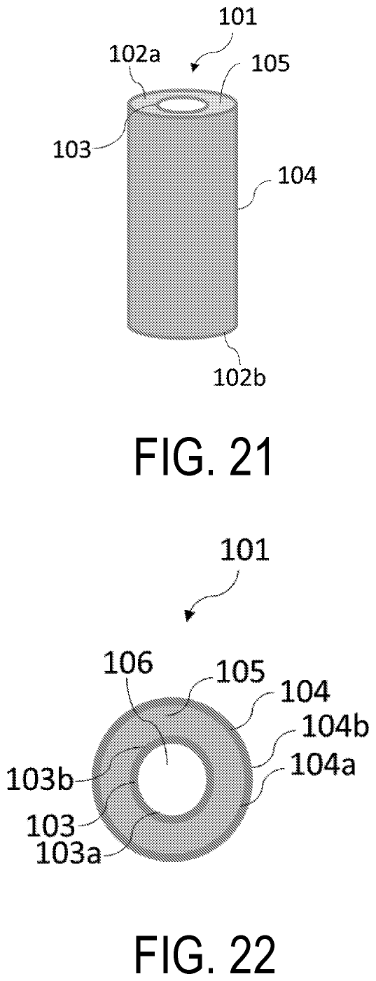

For example, described herein in FIGS. 21 and 22 is a drug delivery device 100 including a cuff 101 having a hollow cylindrical shape with two closed ends (102a and 102b). The cuff including an inner wall 103, an outer wall 104, a lumen 105 extending therethrough, and a composition including an active agent encapsulated within a carrier including a hydrophobic polymer, hydrophilic polymer, amphiphilic polymer, copolymer, or blends thereof present within the lumen 105. The inner wall 103 can include an inner surface 103a and an outer surface 103b and the outer wall 104 can include an inner surface 104a and an outer surface 104b. The inner surface (103a and 104a) of the inner and outer walls (103 and 104) surrounds the lumen 105. The outer surface 103b of the inner wall surrounds a hollow center 106. The inner and outer walls (103 and 104) can include a biodegradable polymer.

In some embodiments, the biodegradable polymer can include a polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, a polyethylene glycol (PEG), gelatin, collagen, elastin, silk fibroin, copolymers thereof, and blends thereof. In some embodiments, natural biodegradable materials (collagen, gelatin, etc.) may be partially or completely crosslinked, e.g., by exposure to glutaraldehyde vapor. In some embodiments, the biodegradable polymer can include polycaprolactone (PCL).

In some embodiments, the inner, outer, or inner and outer wall can further include a non-biodegradable polymer. In some embodiments, the non-biodegradable polymer can include, but is not limited to, polyethylene terephthalate (PET), polyvinyl chloride (PVC), polyethylene (PE), polysulfone (PSU), polyethersulfone (PES), polypropylene (PP), polystyrene (PS), poly(urethanes), poly(acrylates), poly(ethylene vinyl acetate), nylon, copolymers, or blends thereof. In some embodiments, the non-biodegradable polymer can include polyethylene terephthalate (PET).

In some embodiments, the active agent can be encapsulated within a carrier. In some embodiments, the carrier can be a hydrophilic polymer, hydrophobic polymer, amphiphilic polymer (i.e., conjugates of hydrophilic and hydrophobic polymers), co-polymers, or blends thereof. The hydrophilic polymer can be any suitable hydrophilic polysiloxane polymer such as dimethylsiloxane-ethylene oxide block/graft co polymers (e.g., dimethylsiloxane-(25-30% ethylene oxide) block copolymer, dimethylsiloxane-(30-35% ethylene oxide) block copolymer, dimethylsiloxane-(45-50% ethylene oxide) block copolymer, dimethylsiloxane-(50-55% ethylene oxide) block copolymer, dimethylsiloxane-(60-70% ethylene oxide) block copolymer, dimethylsiloxane-acetoxy terminated ethylene oxide block copolymer, dimethylsiloxane-(80% ethylene oxide) block copolymer, dimethylsiloxane-(80-85% ethylene oxide) block copolymer, dimethylsiloxane-(85-90% ethylene oxide) block copolymer), (carbinol functional) methylsiloxane dimethylsiloxane copolymer, (hydroxypropyleneoxypropyl)methylsiloxane-dimethylsiloxane copolymer, hydroxyalkyl terminated poly(propyleneoxy)-polydimethylsiloxane block copolymer, (hydroxyethyleneoxypropylmethylsiloxane)-(3,4-dimethoxyphenylpropyl)methylsiloxane-dimethylsiloxane terpolymer, carbinol (hydroxyl) terminated polydimethylsiloxane, (35% hydroxyethyleneoxypropylmethylsiloxane)-(dimethylsiloxane) copolymer, carboxylate substituted (n-pyrrolidonepropyl)methylsiloxane-dimethylsiloxane copolymers, dimethylaminopropylcarboxamide substituted (n-pyrrolidonepropyl)methylsiloxane-dimethylsiloxane copolymers, tetrahydrofurfuryloxypropylmethylsiloxane, or cyanopropylmethylsiloxane, copolymers, or blends thereof. In some embodiments, the hydrophobic polymer can be any suitable hydrophobic polysiloxane such as linear polydimethylsiloxane (PDMS), polydiethylsiloxane, polydipropylsiloxane, polydihexylsiloxane, polydiphenylsiloxane, cyclosiloxanes, hexamethyldisiloxane, copolymers, or blends thereof. In some embodiments, the amphiphilic polymer can be an amphiphilic polysiloxane polymer such as dodecylmethylsiloxane-hydroxypolyalkyleneoxypropylmethylsiloxane copolymer.

In some embodiments, the cuff can have a length of at least 0.1 cm, (e.g., at least 0.5 cm, at least 1 cm, at least 2 cm, at least 3 cm, at least 4 cm, at least 5 cm, at least 6 cm, at least 7 cm, at least 8 cm, at least 9 cm, at least 10 cm, at least 11 cm, at least 12 cm, at least 13 cm, at least 14 cm, at least 15 cm, at least 16 cm, at least 17 cm, at least 18 cm, or at least 19 cm).

In some embodiments, the cuff can have a length of 20 cm or less, (e.g., 19 cm or less, 18 cm or less, 17 cm or less, 16 cm or less, 15 cm or less, 14 cm or less, 13 cm or less, 12 cm or less, 11 cm or less, 10 cm or less, 9 cm or less, 8 cm or less, 7 cm or less, 6 cm or less, 5 cm or less, 4 cm or less, 3 cm or less, 2 cm or less, 1 cm or less, or 0.5 cm or less).

The cuff can have a length ranging from any of the minimum values described above to any of the maximum values described above. For example, in some embodiments, the cuff can have a length from 0.1 cm to 20 cm, such as from 0.5 cm to 20 cm, from 1 cm to 20 cm, from 5 cm to 20 cm, from 10 cm to 20 cm, from 15 cm to 20 cm, from 0.5 cm to 2 cm, from 0.5 cm to 5 cm, from 0.5 cm to 10 cm, from 1 cm to 10 cm, from 1 cm to 5 cm, from 1 cm to 2 cm, from 5 cm to 10 cm, from 5 cm to 15 cm, or from 1 cm to 3 cm.

In some embodiments, the cuff can have an inner wall diameter of at least 100 μm, (e.g., at least 200 μm, at least 300 μm, at least 500 μm, at least 600 μm, at least 700 μm, at least 800 μm, at least 900 μm, at least 1000 μm, at least 1250 μm, at least 1500 μm, at least 1750 μm, at least 2000 μm, at least 2250 μm, at least 2500 μm, or at least 2750 μm).

In some embodiments, the cuff can have an inner wall diameter of 3000 μm or less, (e.g., 3000 μm or less, 2750 μm or less, 2500 μm or less, 2250 μm or less, 2000 μm or less, 1750 μm or less, 1500 μm or less, 1250 μm or less, 1000 μm or less, 900 μm or less, 800 μm or less, 700 μm or less, 600 μm or less, 500 μm or less, 400 μm or less, 300 μm or less, or 200 μm or less).

The cuff can have an inner wall diameter ranging from any of the minimum values described above to any of the maximum values described above. For example, in some embodiments, the cuff can have an inner wall diameter from 100 μm to 3000 μm, such as from 500 μm to 3000 μm, from 200 μm to 3000 μm, from 100 μm to 2000 μm, from 500 μm to 2000 μm, from 500 μm to 1000 μm, from 200 μm to 1000 μm, from 100 μm to 500 μm, or from 200 μm to 800 μm.

In some embodiments, the cuff can have an outer diameter of at least 50 μm, (e.g., at least 75 μm, at least 80 μm, at least 85 μm, at least 90 μm, at least 95 μm, at least 100 μm, at least 125 μm, at least 150 μm, at least 175 μm, at least 200 μm, at least 225 μm, at least 250 μm, or at least 275 μm).

In some embodiments, the cuff can have an outer diameter of 300 μm or less, (e.g., 275 μm or less, 250 μm or less, 225 μm or less, 200 μm or less, 175 μm or less, 150 μm or less, 125 μm or less, 100 μm or less, or 75 μm or less).

The cuff can have an outer diameter ranging from any of the minimum values described above to any of the maximum values described above. For example, in some embodiments, the cuff can have an outer diameter from 50 μm to 300 μm greater than the inner wall diameter, such as from 50 μm to 100 μm, from 50 μm to 200 μm, from 100 μm to 300 μm, or from 200 μm to 300 μm.

The cuff can have a porosity of at least 5% as determined by mercury porosimetry or apparent density (e.g., at least 10%, at least 20%, at least 30%, at least 40%, at least 50%, or at least 60%). The tubular matrix can have a porosity of 70% or less as determined by mercury porosimetry or apparent density (e.g., 60%6 or less, 50% or less, 40% or less, 30% or less, 20% or less, or 10% or less).

The cuff can have a porosity ranging from any of the minimum values described above to any of the maximum values described above. For example, in some embodiments, the cuff can have a porosity of from 5% to 70% as determined by mercury porosimetry or apparent density (e.g., from 5% to 60%, from 5% to 50%, from 5% to 40%, from 5% to 30%, from 5% to 20%, from 5% to 10%, from 10% to 70%, from 10% to 60%, from 10% to 50%, from 10% to 40% from 10% 30%, from 10% to 20%, from 20% to 30%, from 20% to 40%, from 20% to 50%, from 20% to 60%, from 20% to 70%, from 30% to 70%, from 30% to 60%, from 30% to 50%, from 30% to 40%, from 40% to 70%, from 40% to 60% from 40% to 50%, from 50% to 60%, from 50% 70%, or from 60% to 70%).

The cuff can have a density of at least 0.25 g/c as determined by mercury porosimetry or apparent density (e.g., at least 0.35 g/c, at least 0.45 g/c, or at least 0.65 g/c). The cuff can have a density of 0.70 g/c or less as determined by mercury porosimetry or apparent density (e.g., 0.65 g/c or less, 0.60 g/c or less, 0.55 g/c or less, 0.50 g/c or less, 0.45 g/c or less, 0.40 g/c or less, 0.35 g/c or less, or 0.30 g/c or less).

The cuff can have a density ranging from any of the minimum values described above to any of the maximum values described above. For example, in some embodiments, the cuff can have a density of from 0.25 g/c to 0.70 g/c as determined by mercury porosimetry or apparent density, (e.g., from 0.25 g/c to 0.60 g/c, from 0.25 g/c to 0.50 g/c, from 0.25 g/c to 0.40 g/c, from 0.25 g/c to 0.30 g/c, from 0.3 g/c to 0.60 g/c, from 0.3 g/c to 0.50 g/c, from 0.3 g/c to 0.40 g/c, from 0.35 g/c to 0.60 g/c, from 0.35 g/c to 0.50 g/c, from 0.35 g/c to 0.40 g/c, from 0.4 g/c to 0.60 g/c, from 0.4 g/c to 0.50 g/c, from 0.50 g/c to 0.60 g/c, from 0.50 g/c to 0.70 g/c, from 0.40 g/c to 0.70 g/c, or from 0.30 g/c to 0.70 g/c).

The cuff can have a porosity of at least 5% (e.g., at least 10%, at least 20%, at least 30%, at least 40%, at least 50%, or at least 60%) and a density of at least 0.25 g/c (e.g., at least 0.35 g/c, at least 0.45 g/c, or at least 0.65 g/c) as determined by mercury porosimetry or apparent density.

The cuff can have a porosity of 70% or less (e.g., 60% or less, 50% or less, 40% or less, 30% or less, 20% or less, or 10% or less) and a density of 0.70 g/c or less (e.g., 0.65 g/c or less, 0.60 g/c or less, 0.55 g/c or less, 0.50 g/c or less, 0.45 g/c or less, 0.40 g/c or less, 0.35 g/c or less, or 0.30 g/c or less) as determined by mercury porosimetry or apparent density.

The cuff can have a porosity and a density ranging from any of the minimum values described above to any of the maximum values described above. For example, in some embodiments, the cuff can have a porosity of from 5% to 70% (e.g., from 5% to 60%, from 5% to 50%, from 5% to 40%, from 5% to 30%, from 5% to 20%, from 5% to 10%, from 10% to 70%, from 10% to 60%, from 10% to 50%, from 10% to 40% from 10% 30%, from 10% to 20%, from 20% to 30%, from 20% to 40%, from 20% to 50%, from 20% to 60%, from 20% to 70%, from 30% to 70%, from 30% to 60%, from 30% to 50%, from 30% to 40%, from 40% to 70%, from 40% to 60% from 40% to 50%, from 50% to 60%, from 50% 70%, or from 60% to 70%) and a density of from 0.25 g/c to 0.70 g/c, (e.g., from 0.25 g/c to 0.60 g/c, from 0.25 g/c to 0.50 g/c, from 0.25 g/c to 0.40 g/c, from 0.25 g/c to 0.30 g/c, from 0.3 g/c to 0.60 g/c, from 0.3 g/c to 0.50 g/c, from 0.3 g/c to 0.40 g/c, from 0.35 g/c to 0.60 g/c, from 0.35 g/c to 0.50 g/c, from 0.35 g/c to 0.40 g/c, from 0.4 g/c to 0.60 g/c, from 0.4 g/c to 0.50 g/c, from 0.50 g/c to 0.60 g/c, from 0.50 g/c to 0.70 g/c, from 0.40 g/c to 0.70 g/c, or from 0.30 g/c to 0.70 g/c) as determined by mercury porosimetry or apparent density.

In some embodiments, the cuff can have a pore size of at least 100 nm, (e.g., at least 250 nm, at least 500 nm, at least 750 nm, at least 1 μm, at least 1.5 μm, at least 2 μm, at least 2.5 μm, at least 3 μm, at least 3.5 μm, at least 4 μm, or at least 4.5 μm). In some embodiments, the cuff can have a pore size of 5 μm or less, (e.g., 4.5 μm or less, 4 μm or less, 3.5 μm or less, 3 μm or less, 2.5 μm or less, 2 μm or less, 1.5 μm or less, 1 μm or less, 0.5 μm or less, 250 nm or less, 200 nm or less, or 150 nm or less).

In some embodiments, the cuff can have a pore size of from 100 nm to 5 μm, such as from 100 nm to 4 μm, from 100 nm to 3 μm, from 100 nm to 2 μm, from 100 nm to 1 μm, from 500 nm to 1 μm, from 500 nm to 2 μm, from 500 nm to 3 μm, from 500 nm to 4 μm, from 500 nm to 5 μm, from 1 μm to 2 μm, from 1.5 μm to 2 μm, from 1 μm to 5 μm, from 1 μm to 3 μm, from 1 μm to 4 μm, from 2 μm to 4 μm, from 2 μm to 3 μm, from 2 μm to 5 μm, from 3 μm to 4 μm, from 3 μm to 5 μm, or from 4 μm to 5 μm.

In some embodiments, the drug delivery device releases the active agent over a period of at least 3 days, (e.g., at least 1 week, at least 2 weeks, at least 3 weeks, at least 30 days, at least 3 months, at least 6 months, at least 9 months, or at least 12 months, at least 18 months, at least 2 years, or at least 3 years) when incubated in phosphate buffered saline (PBS) at 37° C.

In some embodiments, the drug delivery device releases the active agent over a period of 3 year or less, (e.g., 2 year or less, 1 year or less, 9 months or less, 6 months or less, 3 months or less, 2 months or less, 30 days or less, or 3 weeks or less, 2 weeks or less, or 1 week or less) when incubated in phosphate buffered saline (PBS) at 37° C.

The drug delivery device releases the active agent over a period ranging from any of the minimum values described above to any of the maximum values described above. For example, the drug delivery device releases the active agent over a period of from 30 days to 3 years, (e.g., from 3 days to 9 months, from 3 days to 6 months, from 3 days to 3 months, from 3 days to 30 days, from 3 days to 2 weeks, from 1 week to 2 weeks, from 1 week to 3 weeks, from 1 week to 30 days, from 1 week to 3 months, from 1 week to 6 months, from 1 week to 9 months, from 1 week to 12 months, from 2 weeks to 3 weeks, from 2 weeks to 30 days, from 2 weeks to 3 months, from 2 weeks to 6 months, from 2 weeks to 9 months, from 2 weeks to 12 months, from 3 weeks to 30 days, from 3 weeks to 3 months, from 3 weeks to 6 months, from 3 weeks to 9 months, from 3 weeks to 12 months, from 30 days to 2 years, from 30 days to 1 year, from 30 days to 9 months, from 30 days to 6 months, or from 30 days to 3 months, from 1 year to 2 years, from 1 year to 3 years, from 2 years to 3 years) when incubated in phosphate buffered saline (PBS) at 37° C.

In some embodiments, the active agent can be in the form of a solid. In some embodiments, the hydrophilic active agent can be in the form of a powder. In some embodiments, the active agent can include an anti-restenosis agent inhibits ingrowth of undesirable cells by preventing dividing, destroying, repelling or preventing adhesion of the undesirable cells. In some embodiments, the anti-restenosis agent can include but is not limited to taxol, a pharmaceutically active taxol derivative, rapamycin, a pharmaceutically active rapamycin derivative, synthetic matrix metalloproteinase inhibitors such as batimastat (BB-94), cell-permeable mycotoxins such as cytochalasin B, gene-targeted therapeutic drugs, c-myc neutrally charged antisense oligonucleotides such as Resten-NG™, nonpeptide inhibitors such as tirofiban, antiallergic drugs such as Rizaben™ (tranilast), gene-based therapeutics such as GenStent™ biologic, heparin, paclitaxel, and any combination of these.

In some embodiments, the active agent as used in the methods described herein may be administered in combination or alternation with additional active agents. Representative examples additional active agents include antimicrobial agents (including antibiotics, antiviral agents and anti-fungal agents), anti-inflammatory agents (including steroids and non-steroidal anti-inflammatory agents), anti-coagulant agents, immunomodulatory agents, anticytokine, antiplatelet agents, and antiseptic agents.

Representative examples of antibiotics include amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, and vancomycin.

Representative examples of antiviral agents include, but are not limited to, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, and zidovudine.

Representative examples of anticoagulant agents include, but are not limited to, heparin, warfarin, rivaroxaban, dabigatran, apixaban, edoxaban, enoxaparin, and fondaparinux.

Representative examples of antiplatelet agents include, but are not limited to, clopidogrel, ticagrelor, prasugrel, dipyridamole, dipyridamole/aspirin, ticlopidine, and eptifibatide.

Representative examples of antifungal agents include, but are not limited to, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, and amphotericin b.

Representative examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Representative examples of non-steroidal anti-inflammatory drugs include ibuprofen, naproxen, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

In some embodiments, the active agent can be present in the composition in an amount from 1 μg/ml to 100,000 μg/ml (e.g., from 1 μg/ml to 50,000 μg/ml, from 1 μg/ml to 10,000 μg/ml, from 1 μg/ml to 1,000 μg/ml, from 1 μg/ml to 250 μg/ml, from 1 μg/ml to 50 μg/ml, from 1 μg/ml to 10 μg/ml, from 1 μg/ml to 5 μg/ml, from 10 μg/ml to 50,000 μg/ml, from 10 μg/ml to 10,000 μg/ml, from 10 μg/ml to 1,000 μg/ml, from 10 μg/ml to 250 μg/ml, from 10 μg/ml to 50 μg/ml, from 50 μg/ml to 50,000 μg/ml, from 50 μg/ml to 10,000 μg/ml, from 50 μg/ml to 1,000 μg/ml, from 50 μg/ml to 250 μg/ml, from 250 μg/ml to 50,000 μg/ml, from 250 μg/ml to 10,000 μg/ml, from 250 μg/ml to 1,000 μg/ml, from 1000 μg/ml to 50,000 μg/ml, from 1000 μg/ml to 10,000 μg/ml, or from 10000 μg/ml to 50,000 μg/ml).

In some embodiments, the cuff can surround a transplant graft, stent, or transplanted artery.

### Methods of Making

Disclosed herein are also methods for preparing a drug delivery device described herein, the method including: forming the inner wall of the cuff on a first rod; forming the outer wall of the cuff on a second rod; removing the porogen from the inner wall, outer wall, or any combination thereof; sintering the inner wall and outer wall; injecting a composition including the active agent encapsulated within a carrier into the lumen of the cuff; sealing the inner wall and the outer wall together to form the drug delivery device. The diameter of the second rod is from 50 μm to 300 μm greater than the diameter of the first rod. Forming the inner wall and outer wall can include electrospinning using a solution of a biodegradable polymer, a porogen, and optionally a non-biodegradable polymer and a voltage difference of from 10 kV to 30 kV, for example from 10 kV to 20 kV, or from 15 kV to 25 kV.

In some embodiments, the method can further include sintering the cuff following forming the outer wall layer. In some embodiments, sintering can include heating at a temperature from 50° C. to 150° C., for example from 90° C. to 110° C. In some embodiments, sintering can include heating for a period from 1 minute to 6 hours, for example from 30 minutes to 6 hours.

In some embodiments, the method can further include washing the cuff following sintering. In some embodiments, the cuff is washed with a saturated sodium bicarbonate solution followed by deionized water. In some embodiments, the porogen is substantially removed from the drug delivery device upon washing with deionized water.

In some embodiments, the method can further include drying the cuff following washing. In some embodiments, drying is in vacuo. In some embodiments, drying is at a temperature of from 50° C. to 150° C., for example from 90° C. to 110° C. In some embodiments, drying occurs for a period from 1 minute to 6 hours, for example from 30 minutes to 6 hours.

In some aspects, the two ends of the cuff are closed. The ends may be closed by any number of sealing techniques as would be appropriately selected by one of skill in the art. In some embodiments, the two ends are sealed using a high frequency tube sealing technique. In such techniques, a high frequency generates an eddy current in the wall, which heats up at least the polymer layers. When the temperature has reached the melting point of the polymer, clamps are closed and the melted polymer is cooled and formed. In some embodiments, the two ends are sealed using hot-jaw tube sealing, where heated jaws apply heat to the outside of the tubular shape to heat up the inside for sealing. In some embodiments, the two ends may be sealed using ultrasonic tube sealing. In such techniques, the polymer composition of the inner layers is heated and melted by high frequency friction force introduced form an ultrasonic horn. Clamps are then closed around the section intended to be sealed, cooled, and formed to seal the ends. In some embodiments, the two ends are sealed using hot air sealing, wherein the system heats the seal area inside the capsule with hot air and then subsequently presses and chills the ends in a subsequent station.

In some embodiments, the biodegradable polymer can include, but is not limited to, a polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, a polyethylene glycol (PEG), gelatin, collagen, elastin, silk fibroin, copolymers thereof, and blends thereof. In some embodiments, natural biodegradable materials (collagen, gelatin, etc.) may be partially or completely crosslinked, e.g., by exposure to glutaraldehyde vapor. In some embodiments, the biodegradable polymer can include polycaprolactone (PCL).

In some embodiments, the inner and/or outer wall can further include a non-biodegradable polymer. In some embodiments, the non-biodegradable polymer can include, but is not limited to, polyethylene terephthalate (PET), polyvinyl chloride (PVC), polyethylene (PE), polysulfone (PSU), polyethersulfone (PES), polypropylene (PP), polystyrene (PS), poly(urethanes), poly(acrylates), poly(ethylene vinyl acetate), nylon, copolymers, or blends thereof. In some embodiments, the non-biodegradable polymer can include polyethylene terephthalate (PET). In some embodiments, the biodegradable polymer, the non-biodegradable polymer, and porogen are present. In some embodiments, the biodegradable polymer, the non-biodegradable polymer, and porogen are present in a ratio of from 70:10:20 to 88:10:2 such as 70:10:20, 80:10:10, 82:10:8, 85:10:5, or 88:10:2 in the solution.

A “porogen” as used herein refers to any material that can be used to create a porous material, e.g. porous polycaprolactone as described herein. In some embodiments, the porogen can include a water-soluble compound, i.e. such that the porogen is substantially removed from the outer layer upon washing the drug delivery device with water. In some embodiments, the porogen can include a soluble organic salt such as HEPES salt; biocompatible soluble inorganic salts such as NaCl or KCl; or any combination there of. In some embodiments, the porogen can include a compound selected from ([Tris(hydroxymethyl)methylamino]propanesulfonic acid) (TAPS), (2-(Bis(2-hydroxyethyl)amino)acetic acid) (Bicine), (Tris(hydroxymethyl)aminomethane) or, (2-Amino-2-(hydroxymethyl)propane-1,3-diol) (Tris), (N-[Tris(hydroxymethyl)methyl]glycine) (Tricine), (3-[N-Tris(hydroxymethyl)methylamino]-2-hydroxypropanesulfonic acid) (TAPSO), (4-(2-hydroxyethyl)-1-piperazineethanesulfonic acid) (HEPES), (2-[[1,3-dihydroxy-2-(hydroxymethyl)propan-2-yl]amino]ethanesulfonic acid) (TES), (3-(N-morpholino)propanesulfonic acid) (MOPS), (Piperazine-N,N′-bis(2-ethanesulfonic acid)) (PIPES), Dimethylarsenic acid, (2-(N-morpholino)ethanesulfonic acid) (MES), or salts thereof, such as the sodium salts thereof.

In other embodiments, the disclosed cuff may be manufactured by any appropriate method as would be readily understood by those of ordinary skill in the art. In some embodiments, the disclosed cuffs may be manufactured by asymmetric membrane formation; a representative example of such methods are provided in Yen, C. et al. “Synthesis and characterization of nanoporous polycaprolactone membranes via thermally- and nonsolvent-induced phase separations for biomedical device application” Journal of Membrane Science 2009, 343:180-88, hereby incorporated herein by reference in its entirety for all purposes. In some embodiments, the disclosed cuffs may be manufactured using three-dimensional printing. In some embodiments, the disclosed cuffs may be manufactured around methylcellulose which is subsequently removed to form the luminal compartment. In some embodiments, the disclosed cuffs may be manufactured by a method described by Envisia Therapeutics in WO 2015/085251, WO 2016/144832, WO 2016/196365, WO 2017/015604, WO 2017/015616, or WO 2017/015675, each of which is hereby incorporated by reference in its entirety for all purposes. In yet other embodiments, the disclosed cuffs may be manufactured by methods similar to those used in the manufacturing of hollow fiber membranes, such as phase inversion including non-solvent induced phase inversion (NIPS), (solvent) evaporation-induced phase inversion (EIPS), vapor sorption-induced phase inversion (VIPS), and thermally induced phase inversion (TIPS) In some embodiments, the disclosed cuffs may be manufacturing using a method similar to the methods described in US 2015/232506, incorporated herein by reference in its entirety for all purposes. In some embodiments, the pores may instead by formed by laser diffraction of the cuffs.

### Methods of Use

Described are methods of treating restenosis in a subject including administering to the subject in need thereof an effective amount of an active agent using a drug delivery device described herein.

Described are also methods of inhibiting intimal hyperplasia in a subject including administering to the subject in need thereof an effective amount of an active agent using a drug delivery device described herein.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### EXAMPLES

### Example 1: Sintered Electrospun Fibers for Engineered Delivery of Low Molecular Weight Compounds

### Abstract

Electrospinning has been used widely for drug delivery due to its versatility and ease of modification of fiber properties. However, total drug loading and release is typically limited by the inherent surface area. Instead a sintering of electrospun fiber to create injectable capsules favoring controlled delivery of low molecular weight compounds was used. As electrospun, the fully resorbable, biocompatible polymer polycaprolactone (PCL) retains its morphology out to 1,042 days of in vitro exposure, illustrating its potential for extended performance. Sintering decreases the pore size by 10- and 28-fold following 56 and 57° C. exposures, respectively. After the 58 and 59° C. exposures, these PCL capsules lose all apparent surface porosity but entrapped pores are observed in the 58° C. cross-section. The use of Rhodamine B (RhB, 479.02 g mol⁻¹), Rose Bengal (RB, 1017.64 g mol-) and albumin-fluorescein isothiocyanate conjugate from bovine serum (BSA-FITC, ˜66,000 g mol⁻¹) as model compounds demonstrated that release (RhB>RB>>BSA-FITC) can be controlled by both molecular weight and available porosity. Interestingly, the ranking of release following sintering was 57>56>59>58° C.; COMSOL simulations explored the effects of capsule wall thickness and porosity on release rate. The model drug adsorption on the available surface area (57 versus 56° C.) and entrapped porosity (59 versus 58° C.) could have also attributed to the observed ranking of release rates. While the 56 and 57° C. exposures allowed the bulk of the release to occur in less than 1 day, the capsules sintered at 58 and 59° C. exhibited release that continued after 12 days of exposure. Controlled confinement of a specific porogen, 4-(2-Hydroxyethyl)piperazine-1-ethanesulfonic acid sodium (HEPES) salt, during spinning followed by sintering enabled zero-order release of RB at rates of 4.26, 22.49 and 33.81 μg/day based on the identity of the carrier fluid.

### INTRODUCTION

Electrospinning has been used widely for drug delivery applications due to its versatility and ease of modification of spun fiber properties. Drug delivery vehicles have been an area of intense interest due to the potential for surface-area based control over release. Delivery has utilized electrospun fibers made from bioabsorbable polymers such as PCL, PLGA, PLA, natural polymers such as chitosan and gelatin and blends of both bioabsorbable and natural polymers. However, net drug loading and release is typically limited by the inherent surface area present in the sample. Sintering of electrospun fiber as a means of creating capsules favoring long-term delivery was used. A polymer solution extruded from a needle or capillary is subjected to a high voltage (˜20-25 kV) electric field. Force balance exists between the surface tension of polymer solution and repulsive force from charging. A charged jet of solution ejects from the tip of the drop to form a “Taylor Cone” at a threshold field strength. The ejected polymer jet is accelerated toward the grounded target while simultaneously drying/shrinking due to solvent evaporation and kinetic forces. Scaling: ˜1 mm to <1 μm. Our prior work involved tube formation by spinning onto a rotating mandrel directed toward vascular applications (Journal of Biomedical Materials Research Part A 2007). The current focus utilizes the same formation technology but directs the result toward controlled drug release.

### Results

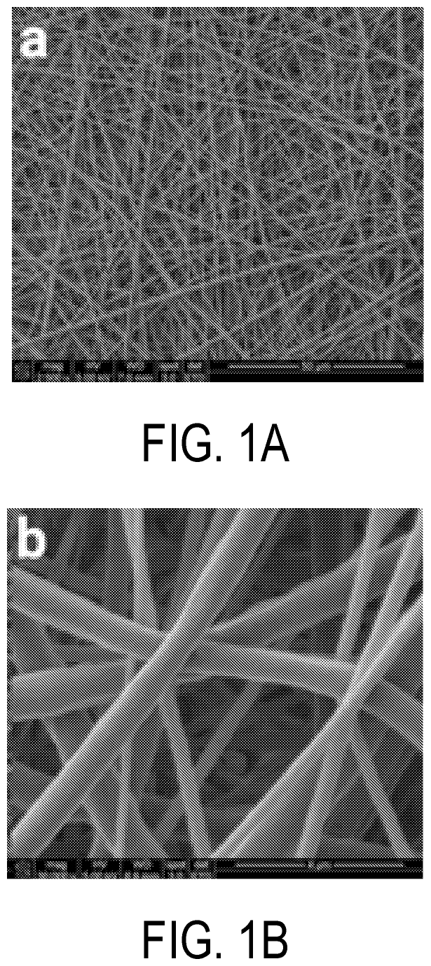

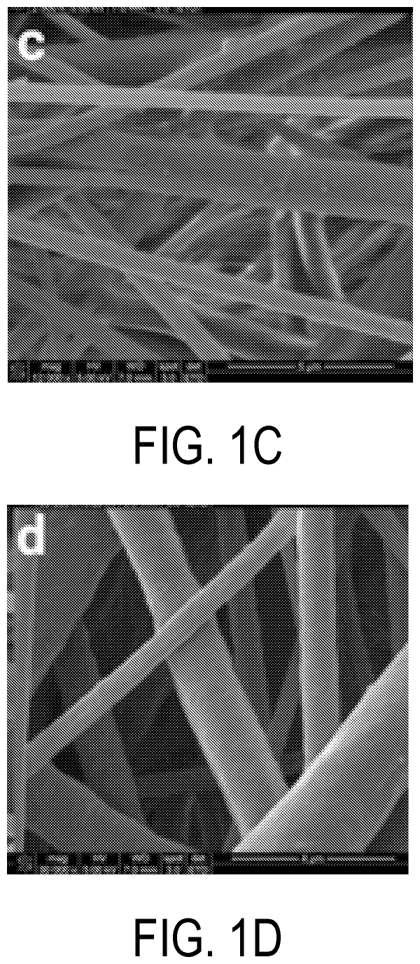

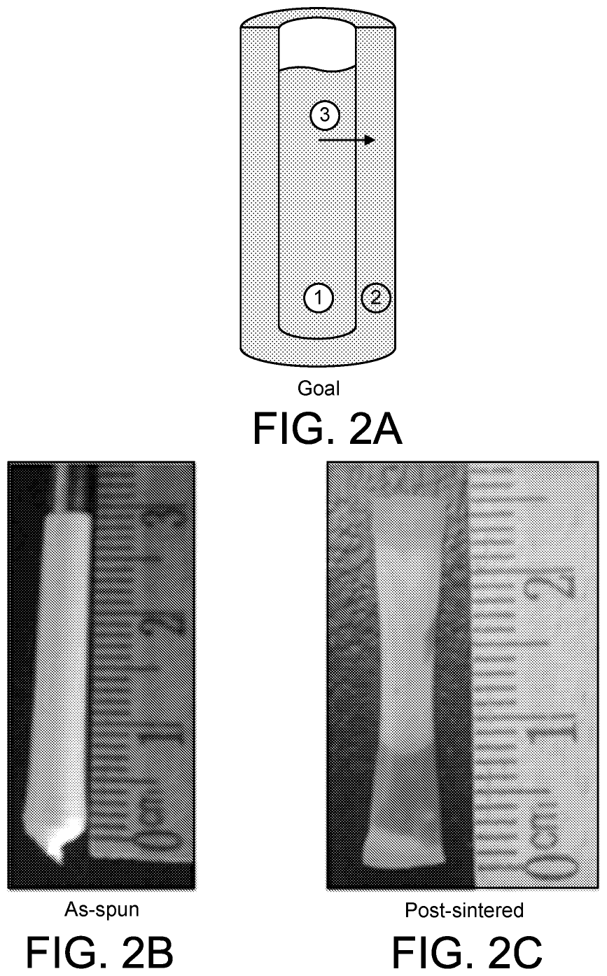

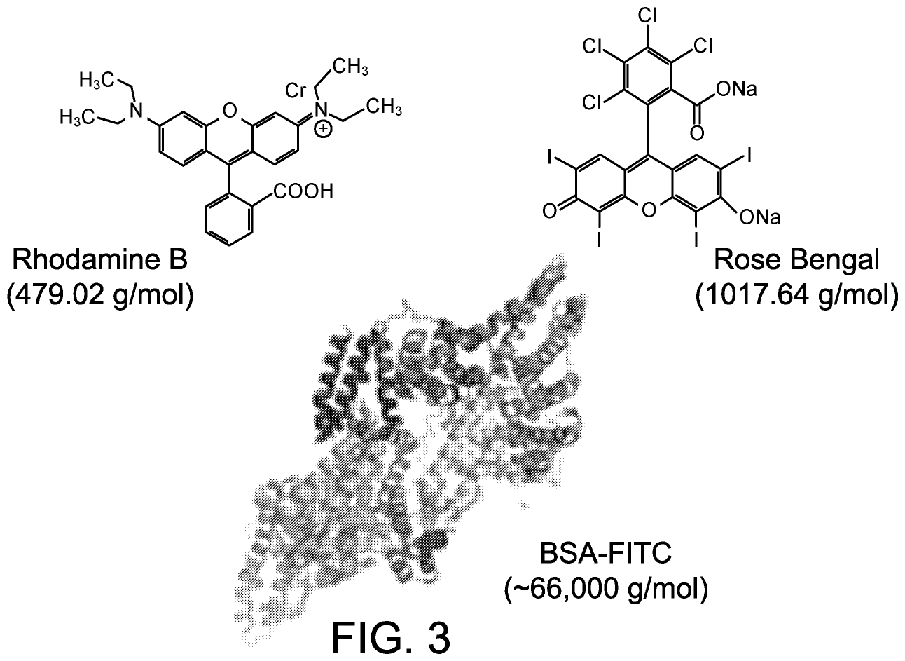

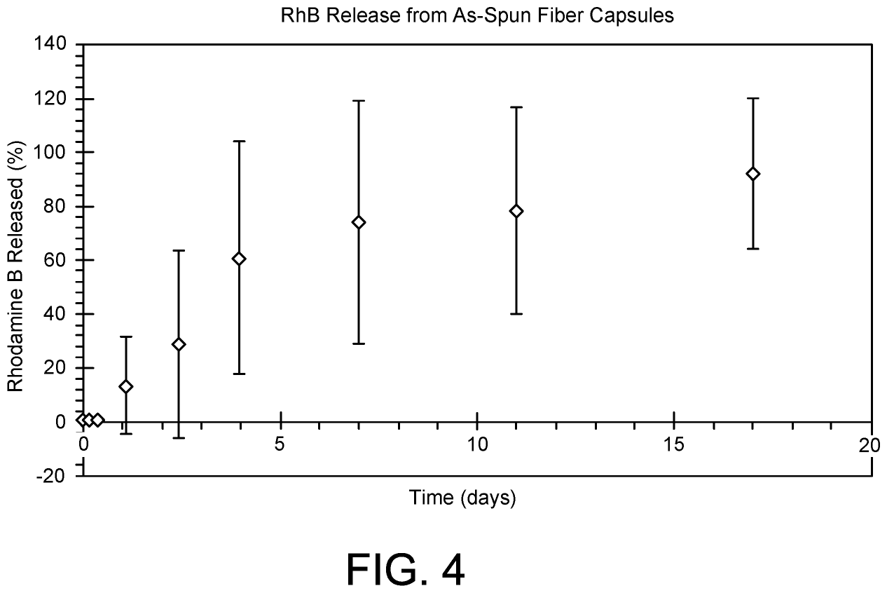

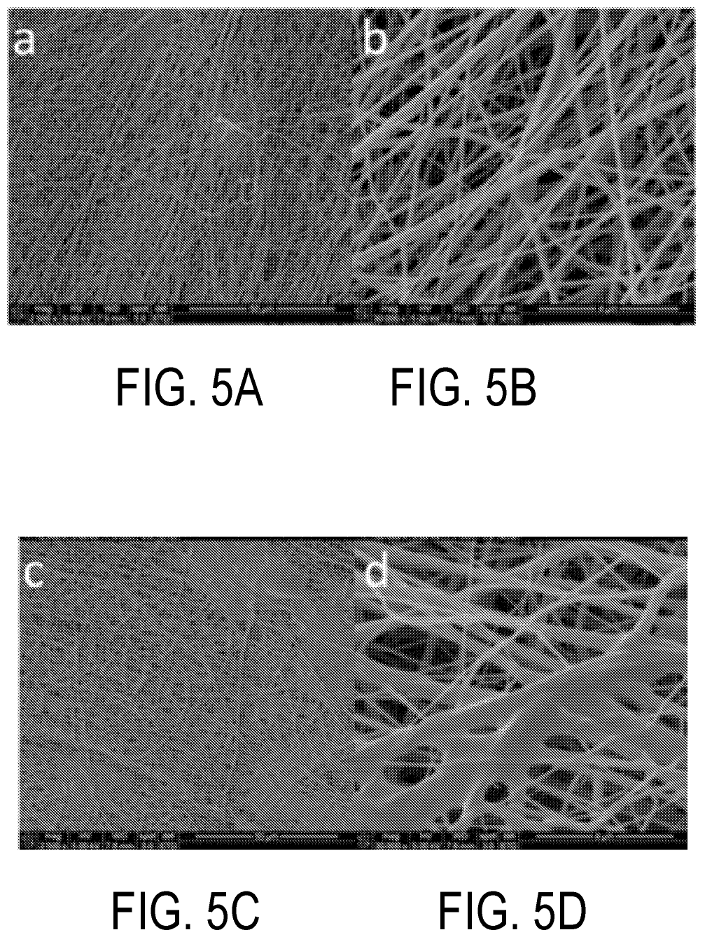

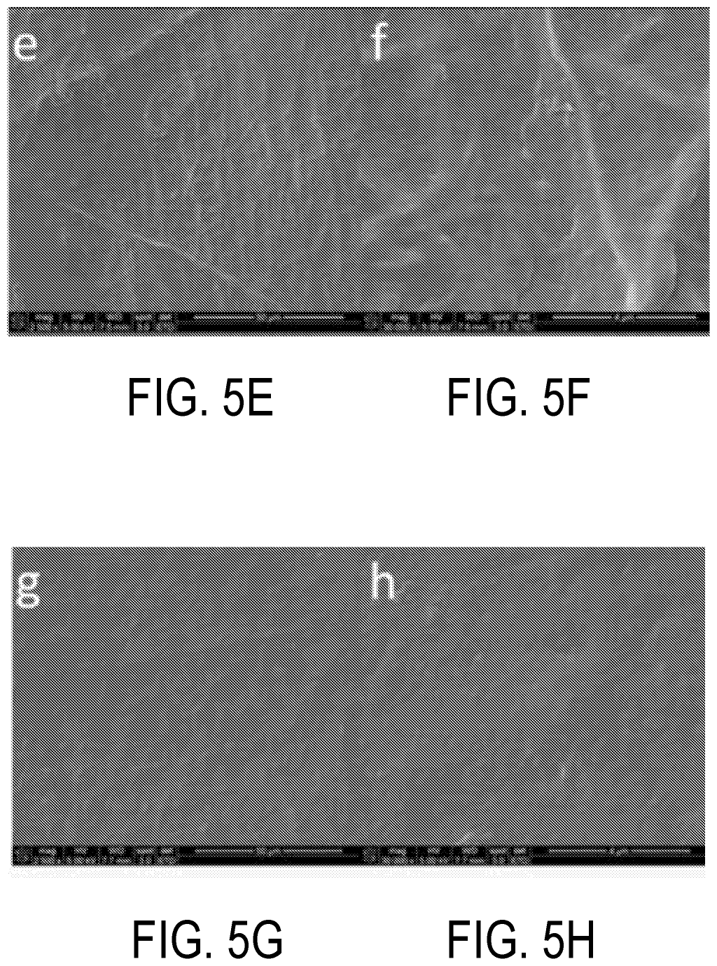

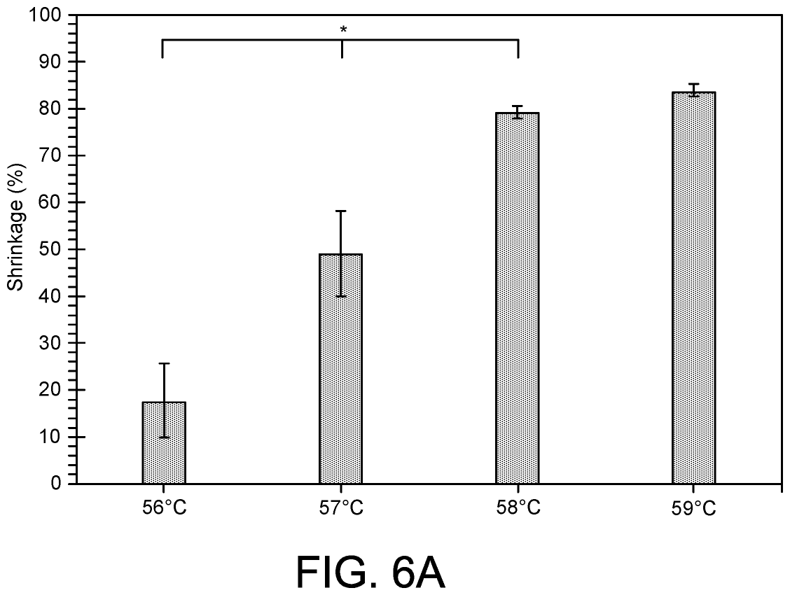

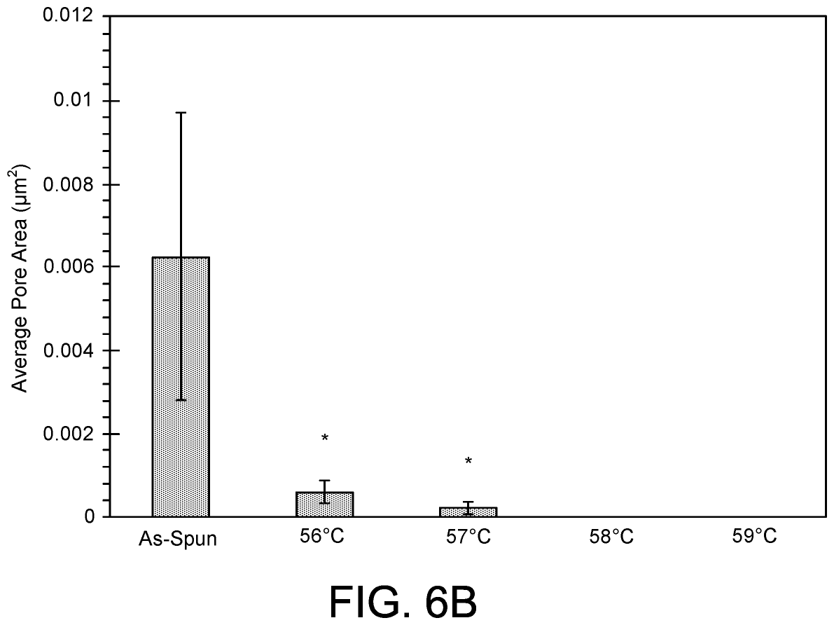

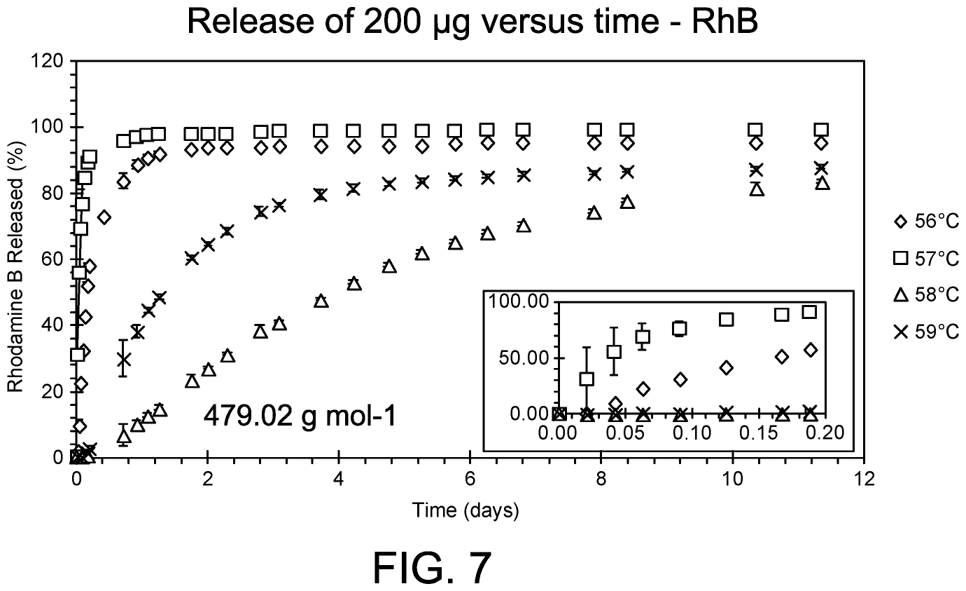

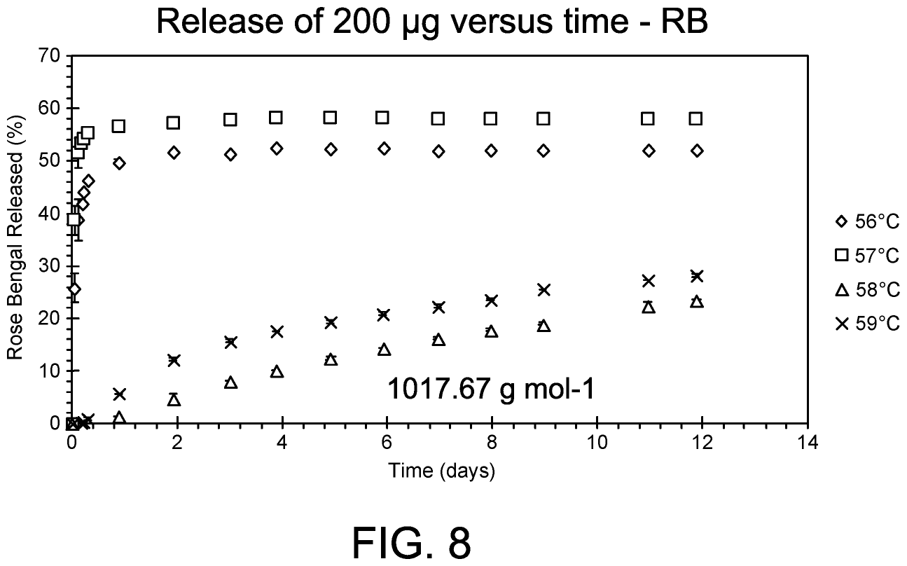

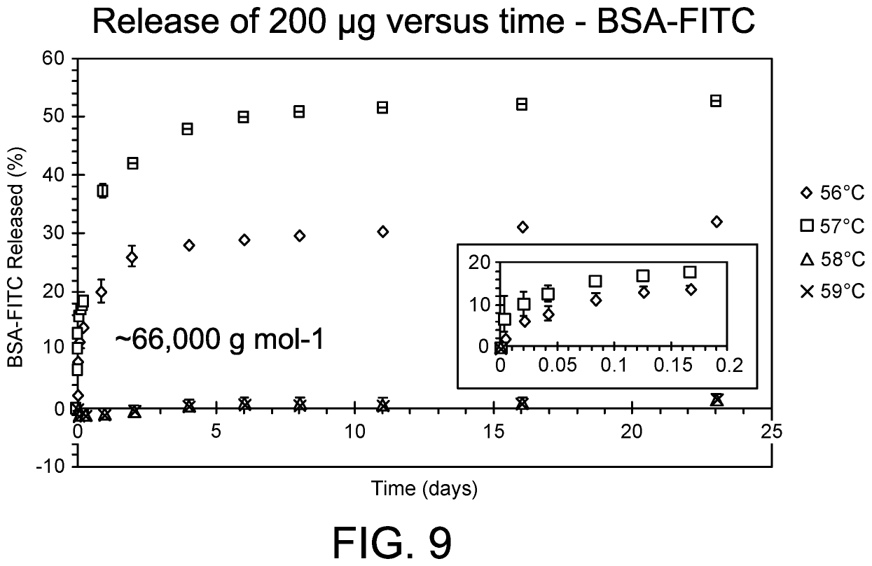

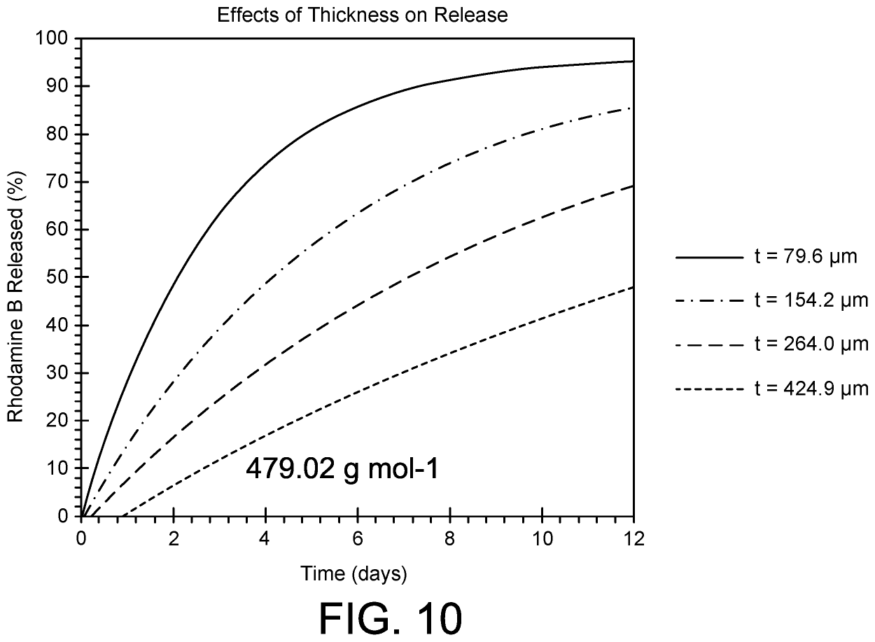

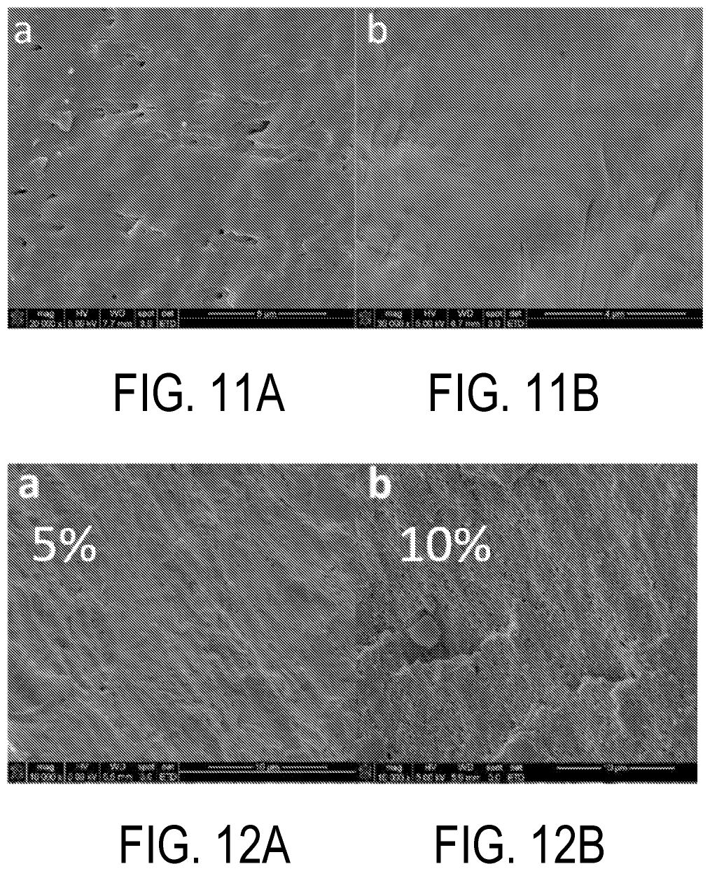

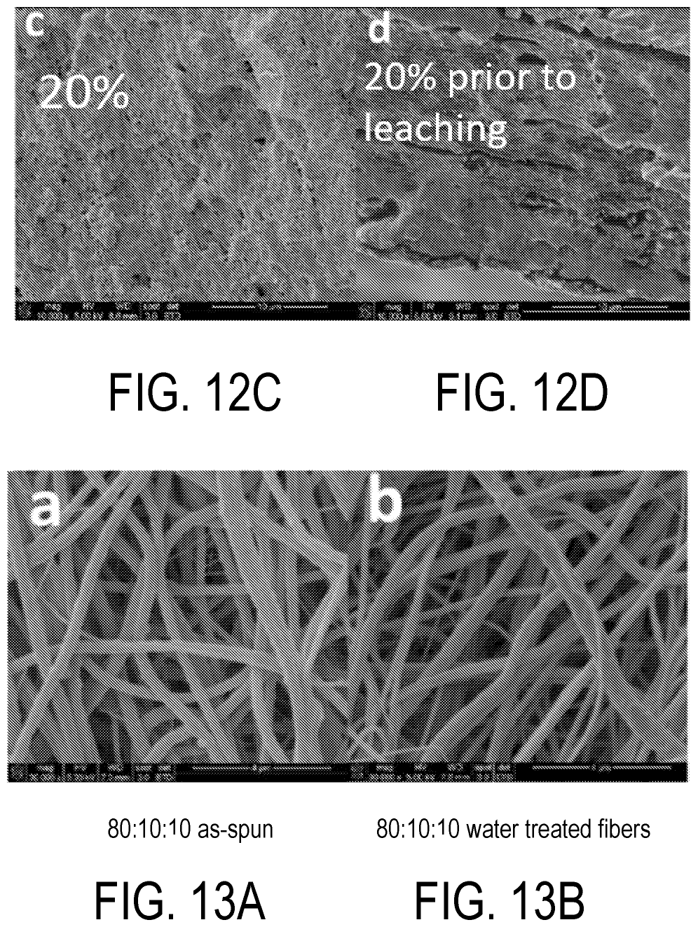

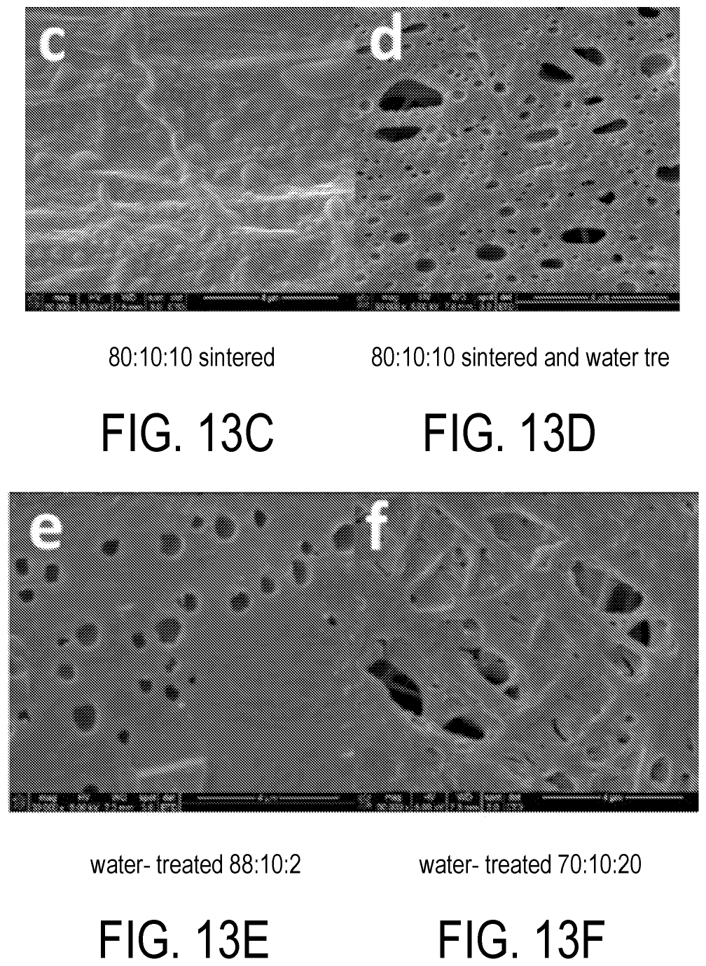

Initial structure post-electrospinning is shown in FIGS. 1A-1D. Capsule formation at scale is shown in FIGS. 2A-2C. Model compounds (Rhodamine B (RhB), Rose Bengal (RB), and BSA-FITC) are shown in FIG. 3. Results for RhB release from as-spun fiber capsules are shown in FIG. 4. FIGS. 5A-5H show images of microstructural transformations at different temperatures (56° C. to 59° C.). Progressive elimination of porosity as temperature increases is shown in FIGS. 6A and 6B. FIG. 7 shows a graph of percent RhB release of 200 μg versus time. Little difference between 56° C. and 57° C. sintering and unsintered fiber˜100% release. At 58° C. sees linear release out to 12 days. At 58° C. and 59° C. release is flipped: faster release at lower porosity. RhB is a relatively small molecule. Intermittent adsorption to surface of remaining porosity greatly slows release. FIG. 8 shows a graph of percent RB release of 200 μg versus time. No condition achieves˜100% release out to 12 days. At 58° C. and 59° C.˜linear release out to 12 days. At 58° C. and 59° C. release are still flipped but not as dramatic: faster release at lower porosity. Intermittent adsorption to surface of remaining porosity continues to slow release but increased molecular weight decreases net differences in diffusion. FIG. 9 shows a graph of percent BSA-FITC release of 200 μg versus time. Much larger molecule needs fully open porosity for any release to occur. Release plateaus quickly. Diffusion through dense PCL is zero. FIG. 10 shows the effects of thickness on percent release of RhB. FIGS. 11A-11B show relatively little porosity remains. This makes a substantial difference in diffusion and surface controlled release. Sequestration within the wall. FIGS. 12A-12D and 13A-13F show controlled salt confinement for controlled porosity using HEPES salt. Table 1 below shows salt extraction efficiency. Extraction was performed using deionized water at 37° C. for 24 hours plus gentle shaking. In most cases extraction was nearly complete. It is likely that Na⁺ was retained and has measurable effects on mechanical properties.

TABLE 1
salt extraction efficiency
Extraction Efficiency

| PCL:PET:HEPES | Weight loss (%) |
|---|---|
| 88:10:2 | 1.66 ± 0.26 |
| 85:10:5 | 4.83 ± 0.30 |
| 82:10:8 | 7.67 ± 0.33 |
| 80:10:10 | 9.93 ± 0.63 |
| 70:10:20 | 25.24 ± 1.72 |

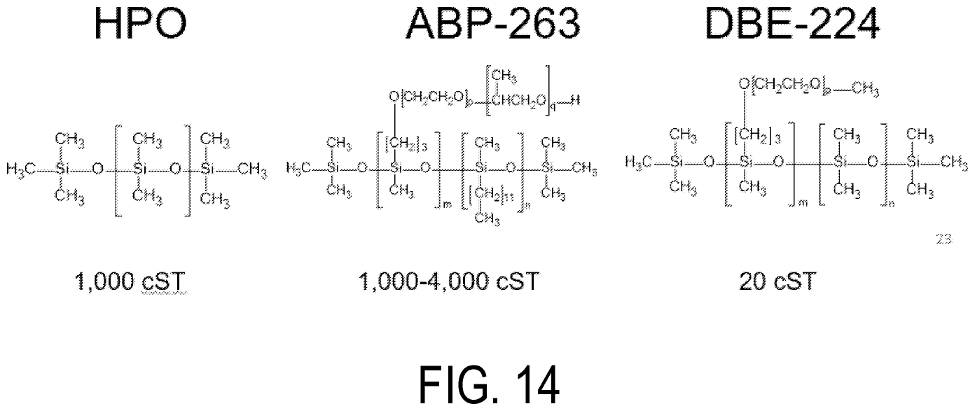

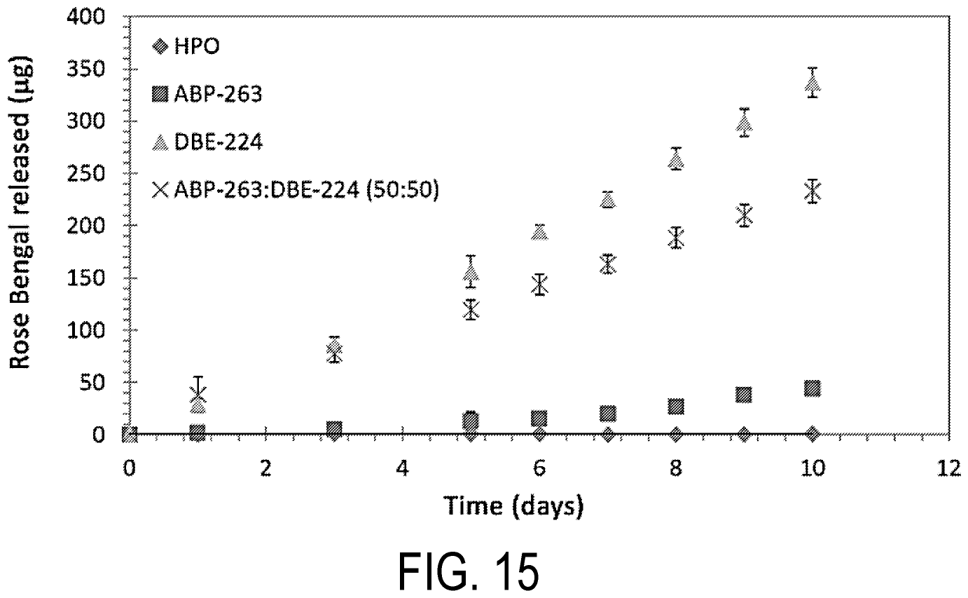

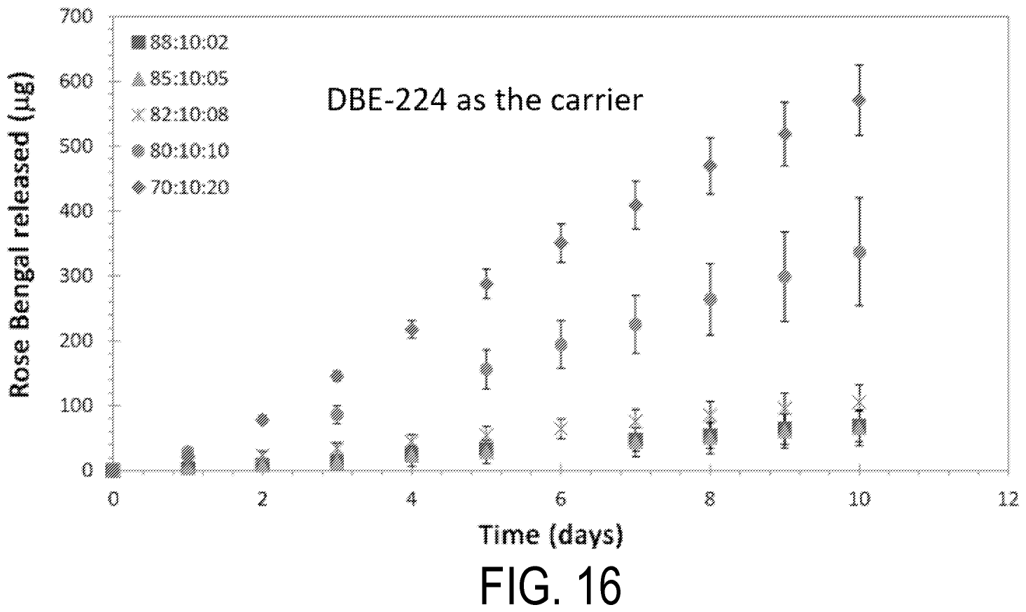

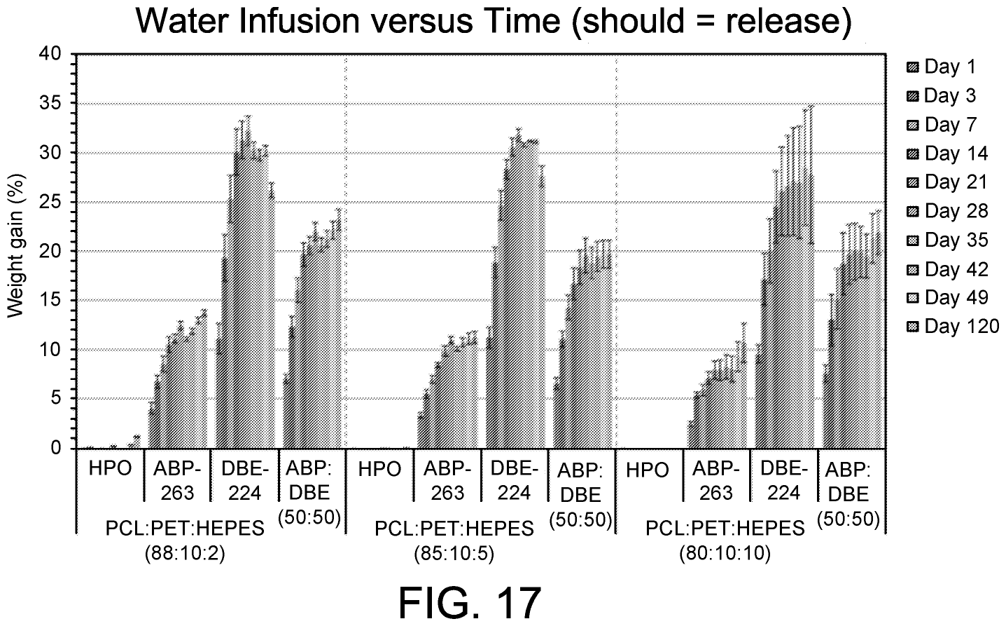

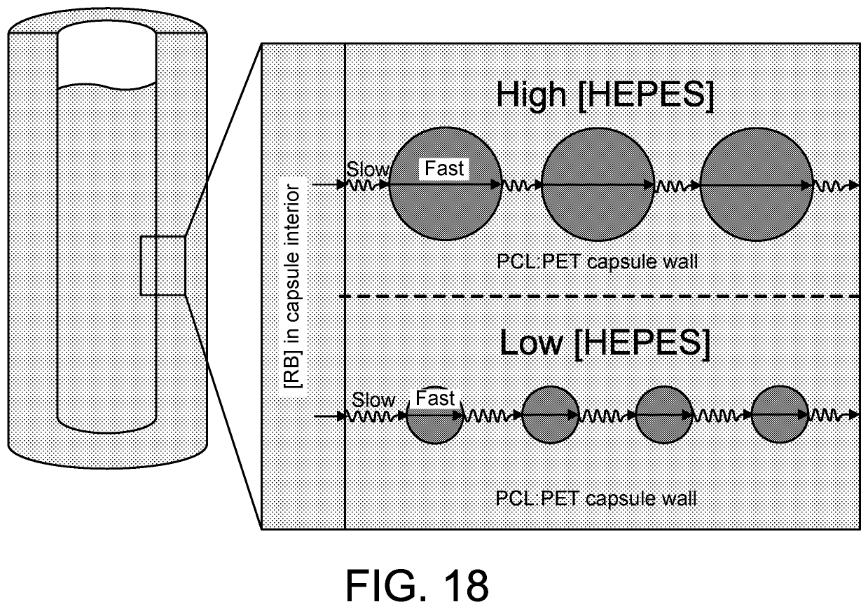

Sample carriers to prevent or minimize hydrolytic degradation are shown in FIG. 14. Less moisture equals less hydrolysis which equals compound remains bioactive for longer periods of time. Silicone oil can be mixed with ‘hydrophilic’ silicone oils to enable some drug solubility. 15 mg (15,000 μg) pressed pellets of RB were used to test four different carriers. Release rate (into PBS) should be slower than from a water carrier but more rapid than pure silicone oil results are shown in FIGS. 15 and 16. Water infusion versus time for different capsules using different carriers is shown in FIG. 17. FIG. 18 shows a conceptual view of release using saltatory diffusion.

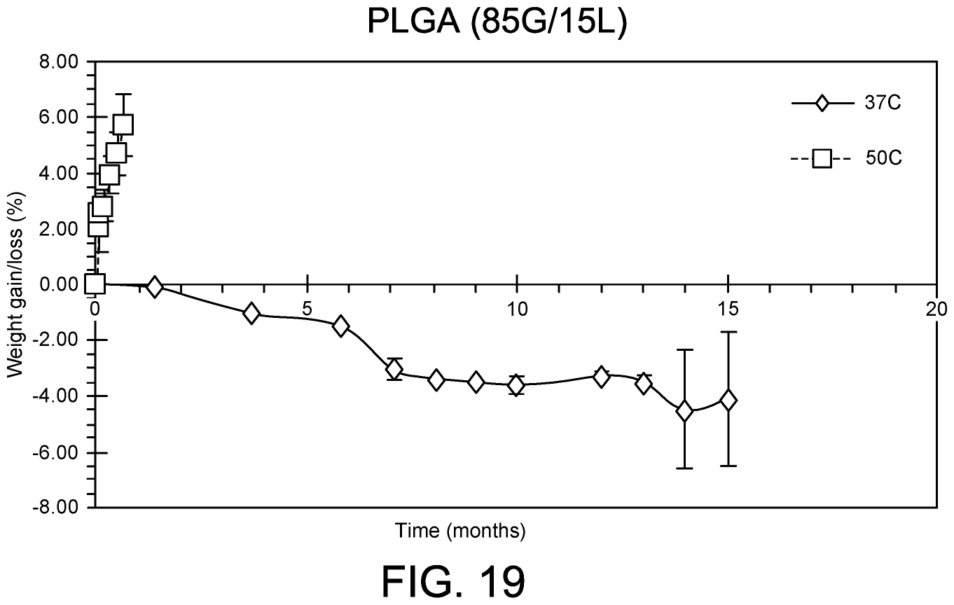

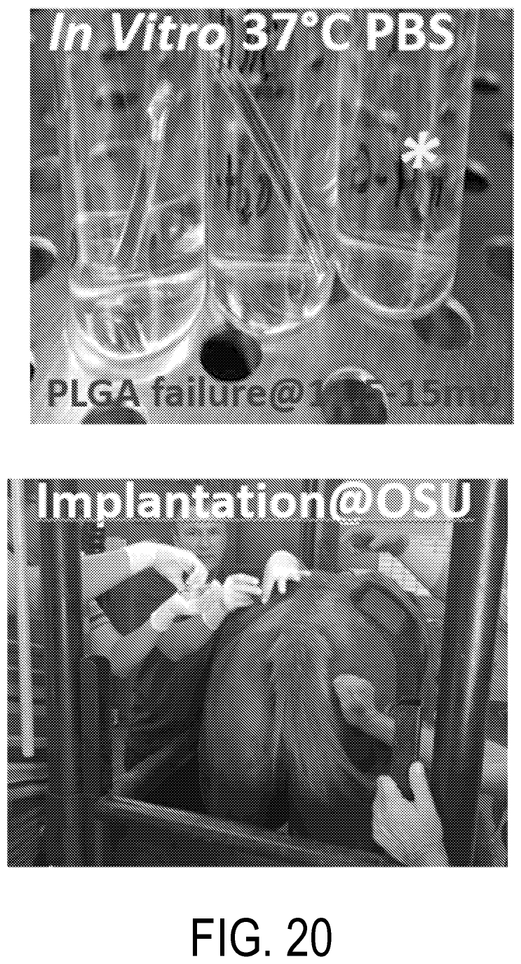

In vitro and In vivo studies were performed (FIG. 19). In vitro studies show that PCL:gelatin 90:10 release for 3 month, 85:15 PLGA release for 1 year, and PCL:PET 75:25 relase for >1 year in PBS.

### CONCLUSION

Incorporation of a soluble salt (HEPES) into the polymer blend (PCL:PET) prior to electrospinning was successful. Water treatment removed the salt and introduced controlled porosity. Zero-order release of a model compound was observed for various PCL:PET:HEPES ratios. Release increased non-linearly versus initial HEPES concentration. COMSOL simulations supported the idea that release is controlled by networks of hydrated discontinuous porosity in which elution occurs in a saltatory fashion.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A drug delivery device comprising:
a cuff having a hollow shape with two closed ends; the cuff comprising an inner wall,
an outer wall,
a lumen extending therethrough, and
a composition comprising an active agent encapsulated within a carrier comprising a hydrophobic polymer, hydrophilic polymer, amphiphilic polymer, copolymer, or blends thereof present within the lumen;
wherein the inner wall and the outer wall comprise an inner surface and an outer surface,
wherein the inner surface of the inner and outer walls surrounds the lumen,
wherein the outer surface of the inner wall surrounds a hollow center,
wherein the inner and outer walls comprise a biodegradable polymer.

2. The device of claim 1, wherein the inner, outer, or inner and outer wall further comprises a non-biodegradable polymer.

3. The device of claim 2, wherein the non-biodegradable polymer comprises polyethylene terephthalate (PET).

4. The device of claim 1 wherein the cuff has a pore size of from 100 nm to 5 μm.

5. The device of claim 1, wherein the active agent is in the form of a solid.

6. The device of claim 1, wherein the cuff has a length of from 0.1 cm to 20 cm.

7. The device of claim 1, wherein the cuff has an inner wall diameter of from 100 μm to 3000 μm.

8. The device of claim 1, wherein the cuff has an outer wall diameter of from 50 μm to 300 μm greater than the inner wall diameter.

9. The device of claim 1, wherein the active agent comprises an anti-restenosis drug.

10. The device of claim 1, wherein the active agent is present in the composition in an amount of from 1 μg/ml to 100,000 μg/ml.

11. The device of claim 1, wherein cuff releases the active agent over a period of at least 30 days, at least 3 months, at least 6 months, at least 9 months, or at least 12 months, at least 18 months, at least 2 years, or at least 3 years when incubated in phosphate buffered saline (PBS) at 37° C.

12. The device of claim 1, wherein the biodegradable polymer comprises a polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, a polyethylene glycol (PEG), gelatin, collagen, elastin, silk fibroin, copolymers thereof, and blends thereof.

13. The device of claim 1, wherein the biodegradable polymer comprises polycaprolactone.

14. The device of claim 1, wherein the cuff surrounds a transplant graft, stent, or transplanted artery.

15. A method for preparing a drug delivery device comprising a cuff having a hollow shape with two closed ends; the cuff comprising an inner wall, an outer wall, a lumen extending therethrough, and a composition comprising an active agent encapsulated within a carrier comprising a hydrophobic polymer, hydrophilic polymer, amphiphilic polymer, copolymer, or blends thereof present within the lumen; wherein the inner and outer wall comprising a biodegradable polymer, and optionally a non-biodegradable polymer, the method comprising:
forming the inner wall of the cuff on a first rod;
forming the outer wall of the cuff on a second rod, wherein the diameter of the second rod is from 50 μm to 300 μm greater than the diameter of the first rod,
wherein forming the inner wall and outer wall comprises electrospinning using a solution of a biodegradable polymer, a porogen, and optionally a non-biodegradable polymer and a voltage difference of from 10 kV to 30 kV;
removing the porogen from the inner wall, outer wall, or any combination thereof,
sintering the inner wall and outer wall;
injecting a composition comprising the active agent encapsulated within a carrier into the lumen of the cuff; and
closing the inner wall and the outer wall together to form the drug delivery device.

16. The method of claim 15, wherein the porogen comprises a soluble organic salt.

17. The method of claim 15, wherein the porogen comprises HEPES salt.

18. The method of claim 15, wherein the biodegradable polymer comprises a polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, a polyethylene glycol (PEG), gelatin, collagen, elastin, silk fibroin, copolymers thereof, and blends thereof.

19. The method of claim 15, wherein the biodegradable polymer comprises polycaprolactone.

20. The method of claim 15, wherein the non-biodegradable polymer comprises polyethylene terephthalate (PET).

21. The method of claim 15, wherein sintering comprises heating at a temperature of from 50° C. to 150° C.

22. The method of claim 15, sintering comprises heating for a period of from 1 minute to 6 hours.

23. A method of treating restenosis in a subject comprising:
administering to the subject in need thereof an effective amount of an active agent using a drug delivery device of claim 1.

24. A method of inhibiting intimal hyperplasia in a subject comprising:
administering to the subject in need thereof an effective amount of an active agent using a drug delivery device of claim 1.
